(19) United States
(12) Patent Application Publication
(10) Pub. No.: US 2005/0129646 A1
(43) Pub. Date: Jun. 16, 2005

(54) Composition capable of forming a polymer matrix comprising hollow reliefs or excrescences

(75) Inventors: Gabin Vic, Venette (FR); Aude Livoreil, Paris (FR); Henri Samain, Bievres (FR); Harry Heinzelmann, Neuchatel (CH); Raphael Pugin, Auvernier (CH); Sylvia Jeney, St. Sulpice (CH)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/990,880

(22) Filed: Nov. 18, 2004

Related U.S. Application Data
(60) Provisional application No. 60/562,554, filed on Apr. 16, 2004.

(30) Foreign Application Priority Data
Nov. 28, 2003 (FR) 0350935

(51) Int. Cl. A61K 7/06; A61K 7/11

(52) U.S. Cl. 424/70.11; 424/70.16; 424/70.17

(57) ABSTRACT

The invention relates to a composition containing n polymers (P₁, P₂, . . . Pₙ) with 10≧n≧2 dissolved in a common solvent (S_{c}) which is liquid at room temperature and at atmospheric pressure, the polymers (P₁, P₂, . . . Pₙ) and the common solvent (S_{c}) being chosen so as to form distinct domains, each containing a single polymer after applying the composition to a human keratin material substrate and subsequently removing the common solvent (S_{c}). Variations thereof.

### REFERENCE TO PRIOR APPLICATIONS

This application claims priority to U.S. provisional application 60/562,554 filed Apr. 16, 2004, and to French patent application 0350935 filed Nov. 28, 2003, both incorporated herein by reference.

### FIELD OF THE INVENTION

The invention relates to compositions capable of forming a polymer matrix comprising hollow reliefs and/or excrescences. The invention also relates to a kit comprising such a composition, to a substrate coated with this composition and to a process comprising the application of this composition.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The term “keratin material” as used herein includes the skin, the nails and keratin fibres. The term “keratin fibres” means the hair, the eyelashes, the eyebrows, the moustache, body hairs and especially head hair. The present invention is particularly directed towards keratin fibres.

The term “keratin material substitute” as used herein means makeup or care accessories made of natural or synthetic material, which serve as replacement accessories (supports) for human keratin material. Among these accessories, mention may be made of false nails, false eyelashes, hairpieces, locks or wigs, or alternatively pastilles or patches that adhere to the skin or the lips (such as beauty spots).

As fashion changes, consumers, who are ever-more demanding, are in search of cosmetic products for the skin or the hair that afford novel or particular cosmetic effects, especially makeup effects. In particular, there is a need for cosmetic products whose application to a support such as human skin or keratin fibres gives rise to a makeup effect different from those of the continuous and uniform films currently obtained with the products available on the market.

In addition, such cosmetic effects, and especially makeup effects, which form a novel relief, giving the keratin materials and especially keratin fibres volume or body, are sought. In addition, the relief should show good adhesion to the made-up support and good fastness, especially with respect to rubbing and impacts.

On the hair, it is sought to form structured reliefs that give, in addition, good cosmetic properties. The reliefs may give coarseness effects, affording styling, a texture or a novel feel, optical effects, such as a colour, a glint or an anti-reflection gloss, or water-repellency effects giving rise to rapid drying.

For certain applications, the desired coating may have, for example, a granitic or “crepe” effect on the support onto which it is applied. Aesthetically attractive effects and/or effects that make it possible to camouflage imperfections, for instance wrinkles, fine lines, spots, red patches or red blotches, may thus be created on the keratin material substrate or substitute. In particular, a contrast effect may be created to dissimulate a defect or turn attention away from an imperfection, or alternatively to modify a surface state.

It may also be sought to form a coating that produces an optically variable image, which may also act as a beauty spot. It may also be sought to create colours solely by an interference phenomenon, optionally without necessarily using dyes that are soluble in the cosmetically acceptable medium or specific pigments, i.e. without posing the difficulties of compatibility, toxicology and regulation encountered during the development of novel compositions comprising dyes or pigments. Thus, the coating used may, without pigment or dye, give rise to the formation of a glint or colour.

For other applications, microreliefs are sought to constitute a diffraction network. In certain cases, an optically variable image is sought to produce a goniochromatic effect or alternatively a holographic effect.

In yet other cases, a change in the wetting or dewetting properties of human keratin materials or substitutes is sought, or, alternatively, a change in their surface to vary the staying power of a care or makeup product.

### DETAILED DESCRIPTION OF THE PREFERRED EMBODIMENTS

The problem solved by the invention is that of providing cosmetic compositions that satisfy better than those of the prior art the various requirements expressed above.

To solve this problem, the invention provides a cosmetic composition comprising, preferably in a physiologically acceptable medium, n polymers (P₁, P₂, . . . Pₙ) with 10≧n≧2 dissolved in a common solvent (S_{c}), the polymers (P₁, P₂, . . . Pₙ) and the common solvent (S_{c}), which is liquid at room temperature and at atmospheric pressure, being chosen so as to form distinct domains, each containing a single polymer, chosen from P₁, P₂, . . . or Pₙ, after applying the cosmetic composition to a human keratin material substrate and subsequently removing the common solvent (S_{c}).

As used herein, the term “each containing a single polymer” and like terms refer to the polymeric component of the domain only, and is intended to denote a situation where only one of the n polymers are present in a particular domain—other components of the composition, that is, components of the composition other than the remaining n−1 polymer(s), may also be present in the domain. In a preferred embodiment a domain consists of a single polymer.

Another subject of the invention concerns a kit comprising (i) a first cosmetic composition comprising, in a physiologically acceptable medium, n polymers (P₁, P₂, . . . Pₙ) with 10≧n≧2 dissolved in a common solvent (S_{c}), the polymers (P₁, P₂, . . . Pₙ) and the common solvent (S_{c}), which is liquid at room temperature and at atmospheric pressure, being chosen so as to form distinct domains, each containing a single polymer, chosen from P₁, P₂, . . . or Pₙ, after applying the cosmetic composition to a human keratin material substrate and subsequently removing the common solvent (S_{c}), and (ii) a second cosmetic composition comprising at least one selective solvent (Sₛ), which is capable of dissolving up to n−1 of the polymers (P₁, P₂, . . . Pₙ) applied to the human keratin material substrate and not dissolving the substrate.

Yet another subject of the invention relates to a human keratin material substrate or a human keratin material substitute, coated with at least one physiologically acceptable polymer matrix comprising hollow reliefs or excrescences, each matrix consisting of a single polymer.

A subject of the invention is also the use of a cosmetic composition comprising, in a physiologically acceptable medium, n polymers (P₁, P₂, . . . Pₙ) with n≧2, dissolved in a common solvent (S_{c}), to form distinct domains, each containing a single polymer, chosen from P₁, P₂, . . . or Pₙ, after applying the cosmetic composition to a human keratin material substrate and subsequently removing the common solvent (S_{c}).

The invention also relates to a cosmetic process comprising the application, to a human keratin material substrate, in particular to the hair, of:
(a) a first cosmetic composition (i) comprising, in a physiologically acceptable medium, n polymers (P₁, P₂, . . . Pₙ) with 10≧n≧2 dissolved in a common solvent (S_{c}), the polymers (P₁, P₂, . . . Pₙ) and the common solvent (S_{c}), which is liquid at room temperature and at atmospheric pressure, being chosen so as to form distinct domains, each containing a single polymer, chosen from P₁, P₂, . . . or Pₙ, and, after applying the cosmetic composition to a human keratin material substrate and subsequently removing the common solvent (S_{c}),
(b) a second cosmetic composition (ii) comprising at least one selective solvent (Sₛ), which is capable of dissolving up to n−1 of the polymers (P₁, P₂, . . . Pₙ) applied to the human keratin material substrate and not dissolving the substrate,
(c) optionally, a third cosmetic composition (iii) comprising at least one polymer (Pₜₕ), before or after (a) or (b) or (d),
(d) optionally also, an additional cosmetic composition for reshaping, dyeing, making up, removing makeup from, protecting, caring for, cleansing or washing human keratin material, this application being made before or after (a) or (b), each of the steps (a), (b), (c) or (d) optionally being followed by rinsing.

Advantageously, the human keratin material substrate is the hair.

Preferably, a number of polymers is used such that n is less than or equal to 5.

Even more preferably, the common solvent (S_{c}) is chosen from water, C₂-C₆ alcohols, C₂-C₆ alkanediols, benzyl alcohol, C₂-C₆ ethers, C₂-C₆ esters, N—(C₁-C₄)alkylpyrrolidones and C₂-C₆ ketones, and also mixtures of these solvents.

The selective solvent (S_{c}) may generally either form structured reliefs based on holes, or structured reliefs based on pillars.

Advantageously, the polymers (P₁, P₂, . . . Pₙ) each have a number-average molecular mass (weight) ranging from 1 kDa to 1000 kDa; the polymers (P₁, P₂, . . . Pₙ) each have a glass transition temperature (Tg) of greater than or equal to 0° C.

The glass transition temperature corresponds to the temperature at which the amorphous material passes from a glassy solid state to a rubbery state. This temperature may be measured by differential thermal analysis, known as the “DTA” method, or by differential calorimetry, known as the “DSC” method, which stands for Differential Scanning Calorimetry. In particular, the glass transition temperature may be measured by differential calorimetry “DSC” according to ASTM standard D3418-97.

The polymers (P₁, P₂, . . . Pₙ) are advantageously chosen from polystyrene, polystyrenesulfonic acid, C₁-C₁₀ polyalkyl methacrylates, C₁-C₁₀ polyalkyl acrylates, C₁-C₁₀ alkylamines, polyalkyleneamines, polyvinylamines, polyvinylpyridines, polylysines, C₁-C₁₀ hydroxyalkylcelluloses, C₁-C₁₀ hydroxyalkyl guars, C₁-C₁₀ hydroxyalkyltriammonium guar chlorides, C₁-C₁₀ trimethylammonium hydroxyalkylcelluloses, C₁-C₁₀ polyalkylene oxides, polysaccharides, polyacrylic acids, polyacrylamides, poly(meth)acrylamides, polyvinyl alcohols, polyvinyl acetates, polyvinylpyrrolidones, C₁-C₁₀ polyalkyleneimines, C₁-C₁₀ polyacrylamidoalkylsulfonics, polyurethanes, polyesters and polyphosphoric acids.

The relative amount by weight of each polymer (P₁, P₂, . . . Pₙ) in the cosmetic composition advantageously ranges from 0.01% to 50%, preferably from 0.1% to 25% and even more preferably from 0.2% to 10%.

Advantageously, the composition according to the invention comprises n polymers (P₁, P₂, . . . Pₙ) and the weight ratio of each of the polymers relative to the total weight of the cosmetic composition is between 0.01 and 100, preferably between 0.02 and 50 and even more preferably between 0.05 and 20.

The composition according to the invention advantageously comprises two polymers (P₁, P₂) and the weight ratio between the two polymers is between 0.01 and 100, preferably between 0.02 and 50 and even more preferably between 0.05 and 20.

The polymers (P₁, P₂, . . . Pₙ) advantageously comprise polystyrene/C₁-C₁₀ polyalkyl methacrylates, C₁-C₁₀ trimethylammonium hydroxyalkylcelluloses/C₁-C₁₀ polyalkyleneimines and C₁-C₁ hydroxyalkylcelluloses/C₁-C₁₀ trimethylammonium hydroxyalkylcelluloses pairs.

Several methods may be envisaged for the preparation of the compositions used according to the invention, for example optical laser-structuring methods, mechanical pressing methods on a polymer matrix, and physicochemical methods, for instance multi-layer metallization or polymer demixing. According to one preferred embodiment of the invention, polymer demixing is used.

The kit according to the invention advantageously also comprises (iii) a third cosmetic composition comprising at least one polymer (Pₜₕ). (Pₜₕ) is a pretreatment polymer for keratin fibres, which may serve, for example, to improve the adhesion of the compositions used in the process according to the invention or to make the surface of the keratin fibres smoother or more attaching.

This polymer serves to “unify” the surface of the fibre and thus to circumvent the natural variability of the surface state of the hair, to produce the structures more easily. It may be any polymer, provided that it is insoluble in the common solvent S_{c} and also insoluble in the selective solvent Sₛ, and provided that it is chemically inert with respect to the polymers (P₁. . . Pₙ) subsequently applied. The term “chemically inert” means that no covalent bond will be created or destroyed between the polymers. Examples of pretreatment polymers that may be mentioned include polyamines, for instance polyalkyleneimine, polyallylamines, polyamino acids, polysaccharides or polyacrylates, preferably polyethyleneimine or polylysine.

Preferably, the kit according to the invention also comprises an additional composition for reshaping, dyeing, making up, removing makeup from, protecting, cleansing or washing a human keratin material substrate.

In composition (i) of the kit, the common solvent (S_{c}) is advantageously chosen from water, C₂-C₆ alcohols, C₂-C₆ alkanediols, benzyl alcohol, C₂-C₆ ethers, C₂-C₆ esters, N—(C₁-C₄)alkylpyrrolidones and C₂-C₆ ketones, and also a mixture of these solvents.

Preferably, in composition (i) of the kit, the polymers (P₁, P₂, . . . Pₙ) each have a number-average molecular mass ranging from 1 kDa to 1000 kDa; advantageously, the polymers (P₁, P₂, . . . Pₙ) are chosen from polystyrene, polystyrenesulfonic acid, C₁-C₁₀ polyalkyl methacrylates, C₁-C₁₀ polyalkyl acrylates, C₁-C₁₀ alkylamines, polyalkyleneamines, polyvinylamines, polyvinylpyridines, polylysines, C₁-C₁₀ hydroxyalkylcelluloses, C₁-C₁₀ hydroxyalkyl guars, C₁-C₁₀ hydroxyalkyltriammonium guar chlorides, C₁-C₁₀ trimethylammonium hydroxyalkylcelluloses, C₁-C₁₀ polyalkylene oxides, polysaccharides, polyacrylic acids, polyacrylamides, poly(meth)acrylamides, polyvinyl alcohols, polyvinyl acetates, polyvinylpyrrolidones, C₁-C₁₀ polyalkyleneimines, C₁-C₁₀ polyacrylamidoalkylsulfonics, polyurethanes, polyesters and polyphosphoric acids.

Advantageously, in the kit, the relative amount by weight of each polymer (P₁, P₂, . . . Pₙ) in the cosmetic composition (i) ranges from 0.01% to 50%, preferably from 0.1% to 25% and even more preferably from 0.2% to 10%.

According to one advantageous embodiment of the kit, the first composition (i) comprises n polymers (P₁, P₂, . . . Pₙ) and the weight ratio of each of the polymers relative to the total weight of the cosmetic composition is between 0.01 and 100, preferably between 0.02 and 50 and even more preferably between 0.05 and 20.

According to one even more advantageous embodiment of the kit, the polymers (P₁, P₂, . . . Pₙ) comprise polystyrene/C₁-C₁₀ polyalkyl methacrylates, C₁-C₁₀ trimethylammonium hydroxyalkylcelluloses/C₁-C₁₀ polyalkyleneimines and C₁-C₁₀ hydroxyalkylcelluloses/C₁-C₁₀ trimethylammonium hydroxyalkylcelluloses pairs.

Advantageously, the substrate according to the invention has a matrix and reliefs, which each independently comprise at least one polymer chosen from polystyrene, polystyrenesulfonic acid, C₁-C₁₀ polyalkyl methacrylates, C₁-C₁₀ polyalkyl acrylates, C₁-C₁₀ alkylamines, polyalkyleneamines, polyvinylamines, polyvinylpyridines, polylysines, C₁-C₁₀ hydroxyalkylcelluloses, C₁-C₁₀ hydroxyalkyl guars, C₁-C₁₀ hydroxyalkyltriammonium guar chlorides, C₁-C₁₀ trimethylammonium hydroxyalkylcelluloses, C₁-C₁₀ polyalkylene oxides, polysaccharides, polyacrylic acids, polyacrylamides, poly(meth)acrylamides, polyvinyl alcohols, polyvinyl acetates, polyvinylpyrrolidones, C₁-C₁₀ polyalkyleneimines, C₁-C₁₀ polyacrylamidoalkylsulfonics, polyurethanes, polyesters and polyphosphoric acids.

Advantageously, as regards the substrate according to the invention, it is the nails, the eyelashes, the hair or substitutes thereof, these substitutes preferably being chosen from false nails, wigs, locks or false eyelashes.

On the substrate, the relief advantageously comprises randomly distributed or ordered patterns, these patterns advantageously being streaks, pillars or cavities.

The invention also relates to a composition comprising a pair of polymers P₁ and P₂ chosen from polystyrene/C₁-C₁₀ polyalkyl methacrylates, C₁-C₁₀ trimethylammonium hydroxyalkylcelluloses/C₁-C₁₀ polyalkyleneimines and C₁-C₁₀ hydroxyalkylcelluloses/C₁-C₁₀ trimethylammonium hydroxyalkylcelluloses pairs.

The cosmetic compositions of the invention may be used as makeup or care products for keratin fibres, especially for the hair or the eyelashes. For the hair, mention may be made of cosmetic products such as shampoos, rinse-out or leave-in conditioners, permanent-waving, relaxing, smoothing, dyeing or bleaching compositions, or alternatively compositions to be applied before or after dyeing, bleaching, permanent-waving or relaxing the hair. Mention may also be made of styling products, lacquers and any products for non-permanently shaping or conditioning the hair. For the eyelashes, mention may be made of mascaras and also products for lengthening the eyelashes.

The cosmetic compositions of the invention may be used as makeup and/or care products for the skin, the lips or the nails. Mention may be made of products such as body creams, foundations, tinted facial creams or lipsticks. The care products may be rinse-out or leave-on products for cleansing facial and/or body skin. It may also be a base makeup or care product, for instance moisturizing care products to be applied before the makeup product itself or products to facilitate the spreading or adhesion of makeup.

The cosmetic composition according to the invention may also contain at least one adjuvant chosen from silicones in soluble, dispersed or micro-dispersed form, nonionic, anionic, cationic and amphoteric surfactants, ceramides, glycoceramides and pseudoceramides, vitamins and provitamins, including panthenol, plant, animal, mineral and synthetic oils, waxes other than ceramides, glycoceramides and pseudoceramides, water-soluble and liposoluble, silicone-based or non-silicone-based sunscreens, mineral and organic, coloured or uncoloured pigments, dyes that are soluble in the cosmetically acceptable medium, nacreous agents and opacifiers, sequestering agents, plasticizers, solubilizers, acidifying agents, basifying agents, mineral and organic thickeners, antioxidants, hydroxy acids, penetrating agents, fragrances and preserving agents.

Needless to say, a person skilled in the art will take care to select this or these additional compound(s), and/or the amount thereof, such that the advantageous properties of the composition according to the invention are not, or are not substantially, adversely affected by the envisaged addition.

Each composition according to the invention may be prepared by a person skilled in the art on the basis of his general knowledge and according to the state of the art, in view of this disclosure.

The compositions in the kits according to the invention are packaged in separate compartments, containers or devices, optionally accompanied by suitable application means, which may be identical or different, such as fine brushes, coarse brushes or sponges.

The invention is illustrated in greater detail in the examples that follow.

### EXAMPLES

The polystyrene and the polymethyl methacrylate have a molecular weight of 100 000.

### Example 1

Composition

| | | |
|---|---|---|
| polystyrene | 2 | g |
| polymethyl methacrylate | 1 | g |
| toluene | qs 100 | g |

The common solvent S_{c} in this case is toluene.

The composition is applied to locks and then dried.

The locks are then treated with a selective solvent, Sₛ, which in this case is cyclohexane, which can dissolve only the polystyrene.

Observation by AFM reveals that the hair is covered with structures of nanometric size (pillars).

### Example 2

Composition

| | | |
|---|---|---|
| polystyrene | 2 | g |
| polymethyl methacrylate | 1 | g |
| toluene | qs 100 | g |

The common solvent S_{c}. in this case is toluene.

The composition is applied to locks and then dried.

The locks are then treated with a selective solvent, Sₛ, which in this case is acetic acid, which can dissolve only the polymethyl methacrylate.

Observation by AFM reveals that the hair is covered with structures of nanometric size (hollows).

### Example 3

Composition 1

| | | |
|---|---|---|
| Pₜₕ: polyethyleneimine Lupasol P ® from BASF | 10 | g |
| water | qs 100 | g |

Composition 2

| | | |
|---|---|---|
| polystyrene | 2 | g |
| polymethyl methacrylate | 1 | g |
| toluene | qs 100 | g |

The common solvent S_{c} in this case is toluene.

Composition 1 is applied to locks and then dried.

Composition 2 is applied to locks and then dried.

The locks are then treated with a selective solvent, Sₛ, which in this case is cyclohexane, which can dissolve only the polystyrene.

Observation by AFM reveals that the hair is covered with structures of nanometric size (pillars).

The above written description of the invention provides a manner and process of making and using it such that any person skilled in this art is enabled to make and use the same, this enablement being provided in particular for the subject matter of the appended claims, which make up a part of the original description and including a composition comprising, in a physiologically acceptable medium, n polymers (P₁, P₂, . . . Pₙ) with 10≧n≧2 dissolved in a common solvent (S_{c}) which is liquid at room temperature and at atmospheric pressure, the polymers (P₁, P₂, . . . Pₙ) and the common solvent (S_{c}) being chosen so as to form distinct domains, each containing a single polymer, chosen from P₁, P₂, . . . or Pₙ, after applying the cosmetic composition to a human keratin material substrate and subsequently removing the common solvent (S_{c}). Also fully described and enabled herein is a kit comprising (i) a first composition comprising, in a physiologically acceptable medium, n polymers (P₁, P₂, . . . Pₙ) with 10≧n≧2 dissolved in a common solvent (S_{c}) which is liquid at room temperature and at atmospheric pressure, the polymers (P₁, P₂, . . . Pₙ) and the common solvent (S_{c}) being chosen so as to form distinct domains, each containing a single polymer, chosen from P₁, P₂, . . . or Pₙ, after applying the cosmetic composition to a human keratin material substrate and subsequently removing the common solvent (S_{c}), and (ii) a second composition comprising at least one selective solvent (Sₛ), which is capable of dissolving at least one and up to n−1 of the polymers (P₁, P₂, . . . Pₙ) in said first composition applied to the human keratin material substrate and not dissolving the substrate. As used above, the phrases “selected from the group consisting of,” “chosen from,” and the like include mixtures of the specified materials.

As used herein, where a certain polymer is noted as being “obtained from” or “comprising”, etc. one or more monomers (or monomer units) this description is of the finished polymer material itself and the repeating units therein that make up, in whole or part, this finished product. One of ordinary skill in the art understands that, speaking precisely, a polymer does not include individual, unreacted “monomers,” but instead is made up of repeating units derived from reacted monomers.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in a physiologically acceptable medium, n polymers (P₁, P₂, . . . Pₙ) with 10≧n≧2 dissolved in a common solvent (S_{c} ) which is liquid at room temperature and at atmospheric pressure, the polymers (P₁, P₂, . . . Pₙ) and the common solvent (S_{c}) being chosen so as to form distinct domains, each containing a single polymer, chosen from P₁, P₂, . . . or Pₙ, after applying the cosmetic composition to a human keratin material substrate and subsequently removing the common solvent (S_{c}).

2. The composition according to claim 1, wherein the human keratin material substrate is hair.

3. The composition according to claim 1, wherein n is less than or equal to 5.

4. The composition according to claim 1, wherein the common solvent (S_{c}) is selected from the group consisting of water, C₂-C₆ alcohols, C₂-C₆ alkanediols, benzyl alcohol, C₂-C₆ ethers, C₂-C₆ esters, N—(C₁-C₄)alkylpyrrolidones and C₂-C₆ ketones, and mixtures thereof.

5. The composition according to claim 1, wherein the polymers (P₁, P₂, . . . Pₙ) each independently have a number-average molecular weight ranging from 1 kDa to 1000 kDa.

6. The composition according to claim 1, wherein the polymers (P₁, P₂, . . . Pₙ) each independently have a glass transition temperature (Tg) of greater than or equal to 0° C.

7. The composition according to claim 1, wherein the polymers (P₁, P₂, . . . Pₙ) are selected from the group consisting of polystyrene, polystyrenesulfonic acid, C₁-C₁₀ polyalkyl methacrylates, C₁-C₁₀ polyalkyl acrylates, C₁-C₁₀ alkylamines, polyalkyleneamines, polyvinylamines, polyvinylpyridines, polylysines, C₁-C₁₀ hydroxyalkylcelluloses, C₁-C₁₀ hydroxyalkyl guars, C₁-C₁₀ hydroxyalkyltriammonium guar chlorides, C₁-C₁₀ trimethylammonium hydroxyalkylcelluloses, C₁-C₁₀ polyalkylene oxides, polysaccharides, polyacrylic acids, polyacrylamides, poly(meth)acrylamides, polyvinyl alcohols, polyvinyl acetates, polyvinylpyrrolidones, C₁-C₁₀ polyalkyleneimines, C₁-C₁₀ polyacrylamidoalkylsulfonics, polyurethanes, polyesters and polyphosphoric acids.

8. The composition according to claim 1, wherein the relative amount by weight of each polymer (P₁, P₂, . . . Pₙ) in the composition is from 0.01% to 50%.

9. The composition according to claim 1, wherein the weight ratio of each of the polymers relative to the total weight of the composition is 0.05-20.

10. The composition according to claim 1, comprising two polymers (P₁, P₂) and the weight ratio between the two polymers is 0.05-20.

11. The composition according to claim 7, wherein the polymers (P₁, P₂, . . . Pₙ) are selected from the group consisting of polystyrene/C₁-C₁₀ polyalkyl methacrylates, C₁-C₁₀ trimethylammonium hydroxyalkylcelluloses/C₁-C₁₀ polyalkyleneimines and C₁-C₁₀ hydroxyalkylcelluloses/C₁-C₁₀ trimethylammonium hydroxyalkylcelluloses pairs.

12. A kit comprising (i) a first composition comprising, in a physiologically acceptable medium, n polymers (P₁, P₂, . . . Pₙ) with 10≧n≧2 dissolved in a common solvent (S_{c}) which is liquid at room temperature and at atmospheric pressure, the polymers (P₁, P₂, . . . Pₙ) and the common solvent (S_{c}) being chosen so as to form distinct domains, each containing a single polymer, chosen from P₁, P₂, . . . or Pₙ, after applying the cosmetic composition to a human keratin material substrate and subsequently removing the common solvent (S_{c}), and (ii) a second composition comprising at least one selective solvent (Sₛ), which is capable of dissolving at least one and up to n-1 of the polymers (P₁, P₂, . . . Pₙ) in said first composition applied to the human keratin material substrate and not dissolving the substrate.

13. The kit according to claim 12, further comprising (iii) a third cosmetic composition comprising at least one polymer (Pₜₕ).

14. The kit according to claim 12, further comprising at least one additional composition for reshaping, dyeing, making up, removing makeup from, protecting, cleansing or washing a human keratin material substrate.

15. The kit according to claim 12, wherein the common solvent (S_{c}) is selected from the group consisting of water, C₂-C₆ alcohols, C₂-C₆ alkanediols, benzyl alcohol, C₂-C₆ ethers, C₂-C₆ esters, N—(C₁-C₄)alkylpyrrolidones and C₂-C₆ ketones, and mixtures thereof.

16. The kit according to claim 12, wherein the polymers (P₁, P₂, . . . Pₙ) each independently have a number-average molecular weight of 1 kDa-1000 kDa.

17. The kit according to claim 12, wherein the polymers (P₁, P₂, . . . Pₙ) are selected from the group consisting of polystyrene, polystyrenesulfonic acid, C₁-C₁₀ polyalkyl methacrylates, C₁-C₁₀ polyalkyl acrylates, C₁-C₁₀ alkylamines, polyalkyleneamines, polyvinylamines, polyvinylpyridines, polylysines, C₁-C₁₀ hydroxyalkylcelluloses, C₁-C₁₀ hydroxyalkyl guars, C₁-C₁₀ hydroxyalkyltriammonium guar chlorides, C₁-C₁₀ trimethylammonium hydroxyalkylcelluloses, C₁-C₁₀ polyalkylene oxides, polysaccharides, polyacrylic acids, polyacrylamides, poly(meth)acrylamides, polyvinyl alcohols, polyvinyl acetates, polyvinylpyrrolidones, C₁-C₁₀ polyalkyleneimines, C₁-C₁₀ polyacrylamidoalkylsulfonics, polyurethanes, polyesters and polyphosphoric acids.

18. The kit according to claim 12, wherein the relative amount by weight of each polymer (P₁, P₂, . . . Pₙ) in composition (i) is 0.01%-50%.

19. The kit according to claim 12, wherein the first composition (i) comprises n polymers (P₁, P₂, . . . Pₙ) and the weight ratio of each of the polymers relative to the total weight of the cosmetic composition is 0.05-20.

20. The kit according to claim 17, wherein the polymers (P₁, P₂, . . . Pₙ) are selected from the group consisting of polystyrene/C₁-C₁₀ polyalkyl methacrylates, C₁-C₁₀ trimethylammonium hydroxyalkylcelluloses/C₁-C₁₀ polyalkyleneimines ,C₁-C₁₀ hydroxyalkylcelluloses/C₁-C₁₀ trimethylammonium hydroxyalkylcelluloses pairs and mixtures thereof.

21. A human keratin material substrate or human keratin material substitute, coated with at least one physiologically acceptable polymer matrix comprising hollow reliefs or excrescences, each matrix containing a single polymer.

22. A substrate according to claim 21, wherein the matrix and the reliefs each independently comprise at least one polymer selected form the group consisting of polystyrene, polystyrenesulfonic acid, C₁-C₁₀ polyalkyl methacrylates, C₁-C₁₀ polyalkyl acrylates, C₁-C₁₀ alkylamines, polyalkyleneamines, polyvinylamines, polyvinylpyridines, polylysines, C₁-C₁₀ hydroxyalkylcelluloses, C₁-C₁₀ hydroxyalkyl guars, C₁-C₁₀ hydroxyalkyltriammonium guar chlorides, C₁-C₁₀ trimethylammonium hydroxyalkylcelluloses, C₁-C₁₀ polyalkylene oxides, polysaccharides, polyacrylic acids, polyacrylamides, poly(meth)acrylamides, polyvinyl alcohols, polyvinyl acetates, polyvinylpyrrolidones, C₁-C₁₀ polyalkyleneimines, C₁-C₁₀ polyacrylamidoalkylsulfonics, polyurethanes, polyesters and polyphosphoric acids.

23. The substrate according to claim 21, wherein it is a nail, the eyelashes, the hair, or substitutes thereof.

24. The substrate according to claim 23, wherein it is false nails, wigs, locks or false eyelashes.

25. The substrate according to one of claim 21 , wherein the relief comprises randomly distributed or ordered patterns.

26. The substrate according to claim 25, wherein the patterns are streaks, pillars or cavities.

27. A composition comprising a pair of polymers P₁and P₂ selected from the group consisting of polystyrene/C₁-C₁₀ polyalkyl methacrylates, C₁-C₁₀ trimethylammonium hydroxyalkylcelluloses/C₁-C₁₀ polyalkyleneimines and C₁-C₁₀ hydroxyalkylcelluloses/C₁-C₁₀ trimethylammonium hydroxyalkylcelluloses pairs.

28. A method, comprising applying the composition of claim 1 to a human keratin material substrate, and subsequently removing the common solvent (S_{c}).

29. A process comprising the application, to a human keratin material substrate, of:
(a) a first cosmetic composition (i) comprising, in a physiologically acceptable medium, n polymers (P₁, P₂, . . . Pₙ) with 10≧n≧2 dissolved in a common solvent (S_{c}) which is liquid at room temperature and at atmospheric pressure, the polymers (P₁, P₂, . . . Pₙ) and the common solvent (S_{c}) being chosen so as to form distinct domains, each containing a single polymer, chosen from P₁, P₂, . . . or Pₙ, after applying the cosmetic composition to a human keratin material substrate and subsequently removing the common solvent (S_{c}),
(b) a second composition (ii) comprising at least one selective solvent (Sₛ), which is capable of dissolving at least one and up to n−1 of the polymers (P₁, P₂, . . . Pₙ) in (i) applied to the human keratin material substrate and not dissolving the substrate,
(c) optionally, a third composition (iii) comprising at least one polymer (Pₜₕ), before or after (a) or (b) or (d),
(d) optionally also, an additional composition for reshaping, dyeing, making up, removing makeup from, protecting, caring for, cleansing or washing human keratin material, this application being made before or after (a) or (b), each of the steps (a), (b), (c) or (d) optionally being followed by rinsing.
